(19) United States
(12) Patent Application Publication
(10) Pub. No.: US 2026/0311818 A1
(43) Pub. Date: Oct. 8, 2026

(54) FEDERATED RETRIEVAL AUGMENTATION FOR AI-BASED DRUG DISCOVERY AND DEVELOPMENT

(71) Applicant: Stephen Gbejule Odaibo, Sugar Land, TX (US)

(72) Inventor: Stephen Gbejule Odaibo, Sugar Land, TX (US)

(73) Assignee: Deep EigenMatics, Inc., Sugar Land, TX (US)

(21) Appl. No.: 19/680,681

(22) Filed: May 18, 2026

(51) Int. Cl. G16C 20/50; G16C 20/70

(52) U.S. Cl. CPC G16C 20/50; G16C 20/70

(57) ABSTRACT

A system and method for performing an AI-based drug discovery and development task across a plurality of entities in a federation. One or more of the plurality of entities each maintains a respective local non-parametric data store. A coordinator receives a query from a respective one of the plurality of entities, causes a similarity determination between the query and respective non-parametric data of one or more other entities, and transmits at least one retrieved non-parametric data object to the querying entity. The query is augmented with at least a portion of the retrieved non-parametric data and used to perform inference on a global neural network, producing an inference output supporting the task. The query is not directly transferred to any of the other entities, and the respective non-parametric data of any of the other entities is not directly transferred to the querying entity.

### FIELD OF THE INVENTION

The present invention relates generally to Artificial Intelligence (AI) and Machine Learning (ML) methods for drug discovery, design, and development, and specifically to federated learning and retrieval augmented generation in pharmaceutical research and development.

### BACKGROUND OF THE INVENTION

Drug discovery and development is a time-consuming and expensive endeavor, with the cost of bringing a new drug to market often exceeding two billion dollars and spanning more than a decade of effort. Despite this substantial investment, the failure rate of candidate drugs in clinical trials remains high.

Artificial intelligence and machine learning methods hold promise for transforming drug design by enabling in silico identification and optimization of candidate ligands for target receptors and other biological targets of therapeutic interest. The effectiveness of such methods depends critically on the availability of large, diverse, and high-quality training data.

Furthermore, the training of machine learning models at scale is very expensive and takes a long time to complete. The objective of such resource-intensive training is to represent the features captured in the training data as parametric weights of the trained model. These parametric weights are frozen in essence after training, and there are significant constraints on any attempts at modification so as not to destroy or degrade the expensive representations. Furthermore, the frozen weights are not readily amenable to updates over time to incorporate new information.

Retrieval augmented generation is a method that augments large language model input queries with related documents from a datastore, thereby becoming able to access non-parametric information—i.e. via a document datastore, the model gets access to information not encoded in the weights of the language model during training. In particular, the datastore can be readily updated at low cost as new information becomes available, and the language model is able to use such non-parametric information in-context without needing to update the weights of the language model itself. In other words, the information source can be augmented and updated without needing to spend the great amount of time and computational cost required to retrain or fine-tune the model.

Retrieval augmented generation was introduced to the field of large language models in general in the year 2020, see: Guu, K., Lee, K., Tung, Z., Pasupat, P., and Chang, M., 2020. REALM: Retrieval-Augmented Language Model Pre-Training. In Proceedings of the 37th International Conference on Machine Learning (pp. 3929-3938). PMLR., and see: Lewis, P., Perez, E., Piktus, A., Petroni, F., Karpukhin, V., Goyal, N., Küttler, H., Lewis, M., Yih, W. T., Rocktäschel, T., Riedel, S., and Kiela, D., 2020. Retrieval-Augmented Generation for Knowledge-Intensive NLP Tasks. Advances in Neural Information Processing Systems, 33, pp. 9459-9474.

Pharmaceutical companies, biotechnology companies, academic institutions, contract research organizations, and other entities engaged in drug discovery each generate proprietary datasets comprising experimental assay results, high-throughput screening outputs, structural biology observations, computational model outputs, and related information. These datasets are typically siloed within the generating entity because of concerns regarding competitive advantage, data privacy, regulatory compliance, contractual obligations, and the protection of intellectual property.

Federated learning is an approach to collaborative model training in which multiple entities contribute to a shared model without the need to directly share their respective underlying data. In a typical federated learning framework, each entity trains a local model on its own data and communicates attributes of the local model (rather than the data itself) to a coordinator, which aggregates the local models into a global model. Variants of federated learning systems addressing aggregation policy, data heterogeneity, communication efficiency, privacy mechanisms including differential privacy, secure multi-party computation, and homomorphic encryption, and other training-time concerns are known in the art.

There have been a number of instances where retrieval augmented generation techniques were applied to protein language models. For instance, see: RETRIEVAL-AUGMENTED FUSION LANGUAGE MODELS FOR AI-BASED PROTEIN AND DRUG DESIGN; U.S. patent application Ser. No. 19/218,291 and Mahbub, S., Kundu, S. and Xing, E. P., PRISM: Enhancing Protein Inverse Folding through Fine-Grained Retrieval on Structure-Sequence Multimodal Representations. arXiv: 2510.11750, 2025.

Similarly, there have been disclosures of federated learning applied to drug discovery and development, see TRANSPARENCY-WEIGHTED FEDERATED LEARNING FOR AI-BASED DRUG DESIGN AND DEVELOPMENT U.S. patent application Ser. No. 19/656,976; and FEDERATED INFERENCE-TIME CONDITIONING FOR AI-BASED DRUG DISCOVERY AND DEVELOPMENT U.S. patent application Ser. No. 19/662,875; and Heyndrickx, W. et al., MELLODDY: Cross-Pharma Federated Learning at Unprecedented Scale Unlocks Benefits in QSAR without Compromising Proprietary Information. J. Chem. Inf. Model. 2024, 64 (7), 2331-2344.

However, there is a critically unmet need that this disclosure addresses. The need for augmentation from federated external non-parametric data sources, whereby non-parametric data held by participating entities in a federation can be used to augment queries of other entities in the federation without sharing such non-parametric data. This is greatly needed because currently, as new data representing drug discovery and development enters into the possession of a participating entity in the federation after training of a global model, such data is not available for use by the other entities in the federation without directly sharing it.

The inventor is not aware of any federated retrieval augmentation system, prior to the present disclosure, whereby non-parametric data held by participating entities in a drug discovery and development federation can be used to augment queries of other entities in the federation without sharing such non-parametric data.

### OBJECTS OF THE INVENTION

It is an object of this invention to provide a system and method for federated retrieval augmentation for AI-based drug discovery, design, and development across a plurality of entities, wherein each respective one of the plurality of entities is enabled to augment a query of a global neural network with non-parametric data that the respective one of the plurality of entities does not possess but that a different one of the plurality of entities possesses at least in part.

It is further an object of this invention to provide a system and method for federated retrieval augmentation for AI-based drug discovery, design, and development across a plurality of entities, wherein each respective one of the plurality of entities is enabled to obtain (or have a coordinator obtain on its behalf) a similarity score between a query in the possession of that entity and some data object or document not in the possession of that entity but in the possession of a different entity in the federation. Non-limiting examples of how the similarity score may be determined include via inner products of embeddings, via relevance matching, via neural network trained to assign a relevance score, via rules-based matching, via nearest neighbor algorithms, or via search trees.

It is further an object of this invention to provide a system and method for federated retrieval augmentation for AI-based drug discovery, design, and development across a plurality of entities, wherein each respective one of the plurality of entities is enabled to augment a query of a global neural network with non-parametric data that the respective one of the plurality of entities does not possess but that a different one of the plurality of entities possesses at least in part; without the query being shared with any of the entities other than the entity already in possession of the query.

It is further an object of this invention to provide a system and method that accommodates therapeutic, prophylactic, diagnostic, and research uses of compounds, peptides, biologics, or other ligands generated, identified, or optimized via the federated retrieval augmentation system disclosed herein, including in vitro biological activity assessment, in vivo biological activity assessment, and administration to a subject in need thereof.

Yet other objects, advantages, and applications of the invention will be set forth in part in the description that follows and in part will be obvious from the description, or may be learned by practice of the invention.

### SUMMARY OF THE INVENTION

The invention disclosed herein includes a system and method for federated retrieval augmentation for AI-based drug discovery and development across a plurality of entities. As used herein, an “entity” is, by way of non-limiting example, a pharmaceutical company, a biotechnology company, an academic institution, a contract research organization, a contract development and manufacturing organization, a regulatory agency, or any other party possessing proprietary or otherwise-restricted datasets related to drug discovery, design, or development.

As used herein and in the claims, the term “AI-based drug discovery and development task” includes, by way of non-limiting example: generation of candidate ligand representations for a target protein; generation of candidate ligand representations conditioned on a desired biased-signaling profile; generation of candidate ligand representations conditioned on similarity to, or dissimilarity from, a reference ligand; generation of candidate ligand representations conditioned on a multi-receptor selectivity profile; in silico prediction of one or more ligand attributes including without limitation absorption, distribution, metabolism, excretion, and toxicity (ADMET) attributes, binding affinity, functional agonism, functional antagonism, biased agonism, selectivity, pharmacokinetic attributes, and pharmacodynamic attributes; cross-validation of in silico predicted ligand attributes against in vitro or in vivo measurements of corresponding attributes of synthesized ligands; lead selection; lead optimization; scaffold hopping; target identification and druggability assessment; off-target liability assessment; and federated sharing of assay data, screening data, structure-activity-relationship data, in vitro data, and in vivo data via aggregation of attributes of respective local neural networks trained on such data and via retrieval augmentation of input queries to a global neural network on data held by entities in the federation.

In some embodiments, the query is processed to enhance privacy prior to similarity-determining computation on the other entities. Non-limiting examples include: (i) application of differential privacy noise to one or more numerical descriptors associated with a query or with the non-parametric data; (ii) processing of queries via secure multi-party computation across the plurality of entities; (iii) homomorphic encryption of one or more numerical descriptors associated with a query or non-parametric data, wherein computation involving such descriptors is performed under encryption; (iv) k-anonymization of queries and/or non-parametric data; (v) restriction of non-parametric data visibility on a per-entity basis according to entity-specific access policies; (vi) restriction of query visibility on a per-entity basis according to entity-specific access policies; (vii) execution of similarity determination within a trusted execution environment with remote attestation; and (viii) any combination of the foregoing.

A candidate ligand representation produced by the global neural network according to a method or system of the invention disclosed herein may be used as the basis for synthesizing a small-molecule ligand, a peptide ligand, a peptidomimetic, a cyclic peptide ligand, a stapled peptide ligand, a biologic, an antibody, an antibody fragment, a fusion protein, a nucleic-acid-based ligand, an oligonucleotide, an aptamer, or any other ligand modality. The synthesized ligand may be assessed for biological activity in vitro, including without limitation by binding assays, cell-based functional assays, signaling-bias assays, ADMET assays, and toxicology assays; and may be assessed for biological activity in vivo in one or more model organisms or in a human subject. The synthesized ligand may be administered to a subject for therapeutic, prophylactic, diagnostic, or research purposes. In some embodiments, the synthesized ligand is administered as part of a method of treating or preventing a disease or condition characterized by a target receptor conformational state, signaling profile, or molecular signature represented in non-parametric data retrieved from one or more entities of the federation at inference on the global neural network. In some embodiments, the synthesized ligand is used as a companion diagnostic for identifying subjects whose target conformational state, signaling profile, or molecular signature corresponds to a representation in non-parametric data retrieved from one or more entities of the federation at inference, optionally followed by administering a matched therapeutic ligand to the identified subject.

Non-limiting examples of target receptor classes for which the invention disclosed herein is suitable include G-Protein Coupled Receptors (GPCRs), ion channels, enzymes, nuclear receptors, tyrosine kinase receptors, members of an insulin-like receptor family, transporters, cytokine receptors, and Toll-like receptors.

The coordinator may be implemented by, and its functions performed by, any of: a central server; a distributed set of servers; a peer-to-peer protocol executed by the plurality of entities without a central server; a trusted execution environment hosted by one of the plurality of entities or by a third party; a smart contract executed on a blockchain or other distributed ledger; or any combination of the foregoing. The coordinator may further be configured to authenticate the plurality of entities, to enforce access policies governing visibility of federated non-parametric data records, and to maintain audit logs of queries and retrievals.

The invention described herein provides a concrete technological improvement to the field of federated machine learning for AI-based drug discovery and development. Specifically, conventional federated learning systems provide no mechanism for retrieval augmentation based on non-parametric data held by participating entities; whereby a querying entity can augment its query by data held by a different participating entity without that data being directly shared with the querying entity. Prior to this disclosure, querying entities were forced either to perform inference without the relevant non-parametric data, yielding inferior outputs, or to negotiate bilateral data-sharing arrangements with attendant transmission, contractual, regulatory, and competitive risks. The invention disclosed herein solves this technological problem by providing a federated retrieval augmentation and a global neural network, thereby enabling retrieval augmented inference across data-sovereignty boundaries without direct transmission of the query to the other entities and without direct transmission of the non-parametric data to the querying entity. The invention thereby enables accelerated performance of an AI-based drug discovery and development task, including by way of non-limiting example accelerated generation of candidate ligand representations conditioned on receptor conformations, reference ligand structures, multi-receptor selectivity profiles, ADMET profiles, and patient-subgroup signatures held by other entities in the federation; and accelerated in silico cross-validation of attributes of generated candidate ligand representations against reference attributes held by other entities in the federation. The improvement is realized by the specific computational steps set forth in the claims, including the step of receiving a query from a participating entity; performing a similarity determination between the query and non-parametric data held by one or more other entities but not the querying entity-doing so in a manner that neither shares the query directly with any of the one or more other entities nor directly shares relevant non-parametric data of any of the one or more other entities with the querying entity; transmitting one or more similarity-matched data objects or documents to the querying entity; augmenting the query with some of the received non-parametric data and performing inference on the global neural network using the augmented query.

Furthermore, the invention described herein provides a practical application of the herein disclosed federated retrieval augmentation system to drug discovery, design, and development, including by way of method-of-manufacture and method-of-treatment embodiments. A candidate ligand representation produced by the global neural network to which queries are augmented may be used as the basis for synthesizing a corresponding ligand, assessing biological activity of the synthesized ligand in vitro and/or in vivo, and administering the synthesized ligand to a subject for therapeutic, prophylactic, diagnostic, or research purposes. The practical application disclosed herein integrates the abstract aspect, if any, of the federated retrieval augmentation method into a specific drug discovery, design, and development pipeline, and produces concrete physical outputs in the form of synthesized ligands and therapeutic interventions.

In summary, the invention disclosed herein includes a system and method for federated retrieval augmentation for AI-based drug discovery, design, and development; wherein a query held by a querying entity of a federation is augmented with non-parametric data held by one or more other entities of the federation, without direct transmission of the query to the other entities and without direct transmission of the non-parametric data to the querying entity, and wherein inference is performed on a global neural network using the augmented query to produce an output supporting one or more drug discovery and development tasks including, without limitation, candidate ligand generation, lead selection, lead optimization, in silico cross-validation of ligand attributes, and synthesis and administration of a corresponding ligand for therapeutic, prophylactic, diagnostic, or research purposes.

The invention consists of the processes outlined herein and their relation to each other, as well as all modifications that leave the spirit of the invention invariant. The scope of the invention is outlined in the claims section.

### BRIEF DESCRIPTION OF THE DRAWINGS

In the following detailed description of the invention, we reference the drawings, in which:

FIG. 1 is an illustrative example of a G-Protein Coupled Receptor (GPCR), serving as a non-limiting example of a target receptor for which the invention is applicable.

FIG. 2 is a schematic of a centralized federated learning architecture comprising a plurality of entities and a central coordinator.

FIG. 3 is a schematic of an embodiment of a federated retrieval augmentation architecture comprising a plurality of entities, a coordinator, and a retrieval augmentation query path including a query and corresponding retrieved data.

FIG. 4 is an illustrative example of a training architecture of a transformer neural network, by way of non-limiting example, with a sequence head and a structure head, accepting a specified condition comprising a structure input, a residue input, and a property input. The depicted network may be trained at a local training facility of an entity in an embodiment of the federated retrieval augmentation system disclosed herein.

FIG. 5 is an illustrative example of an inference architecture of a transformer neural network, by way of non-limiting example, with a sequence head and a structure head, accepting a specified condition comprising a structure input, a residue input, and a property input. The depicted network embodies a trained global neural network that receives, at inference time, a query augmented in part with non-parametric data retrieved from one or more entities of the federation in accordance with the invention disclosed herein.

FIG. 6 is a schematic of an embodiment of a privacy-layered data processing arrangement, comprising a raw data input (a query or retrieved non-parametric data), a data processor implementing one or more privacy-enhancing mechanisms, and a processed data output for distribution to subscriber entities.

FIG. 7 is an example of a computing environment.

### DETAILED DESCRIPTION OF THE PREFERRED EMBODIMENTS

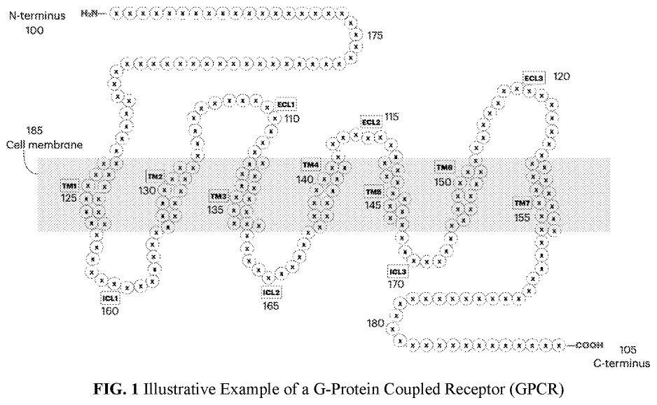

The illustration in FIG. 1 is of a G-Protein Coupled Receptor (GPCR), which serves as a non-limiting example of a target receptor for which the invention is applicable. With reference to FIG. 1, the GPCR comprises an extracellular N-terminus 100 and an intracellular C-terminus 105; three extracellular loops designated ECL1 110, ECL2 115, and ECL3 120; seven transmembrane domains designated TM1 125, TM2 130, TM3 135, TM4 140, TM5 145, TM6 150, and TM7 155; three intracellular loops designated ICL1 160, ICL2 165, and ICL3 170; an N-terminal extracellular chain segment 175; and a C-terminal intracellular chain segment 180. The GPCR, also called a 7 Transmembrane Receptor (7TMR), spans the cell membrane 185 seven times via its seven transmembrane domains. Individual amino acid residues of the receptor are depicted as circles containing an “X” character within the chain. The ligand-generating embodiments of the invention are not limited to GPCR ligands; other non-limiting examples of applicable target receptor classes include ion channels, enzymes, nuclear receptors, tyrosine kinase receptors, and insulin-like receptor families.

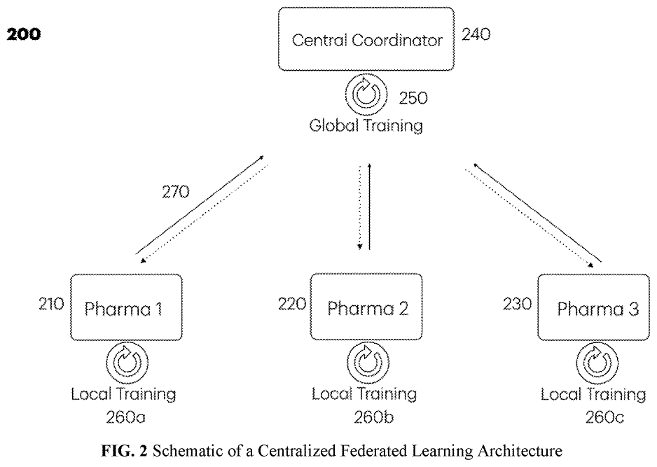

FIG. 2 depicts a centralized federated learning architecture 200 provided herein for context; the invention disclosed herein does not require the global neural network to be trained by the federated learning procedure depicted in FIG. 2. A plurality of entities 210, 220, 230 (any number K≥2 of entities is supported; three are shown for illustrative purposes and are labeled Pharma 1, Pharma 2, and Pharma 3) each possesses a respective local dataset and a respective local training facility 260a, 260b, 260c. A central coordinator 240 is communicatively coupled to each of the plurality of entities via a communication channel 270. The central coordinator 240 comprises a global training facility 250. The entities 210, 220, 230 may be, by way of non-limiting example, pharmaceutical companies, biotechnology companies, academic institutions, contract research organizations, or regulatory agencies. In operation, each respective one of the plurality of entities 210, 220, 230 trains a respective local neural network on its respective local dataset using its respective local training facility 260a, 260b, 260c. Each entity then transmits attributes of its respective local neural network to the central coordinator 240 via the communication channel 270. The central coordinator 240 aggregates the received attributes of the local neural networks into a global neural network using the global training facility 250.

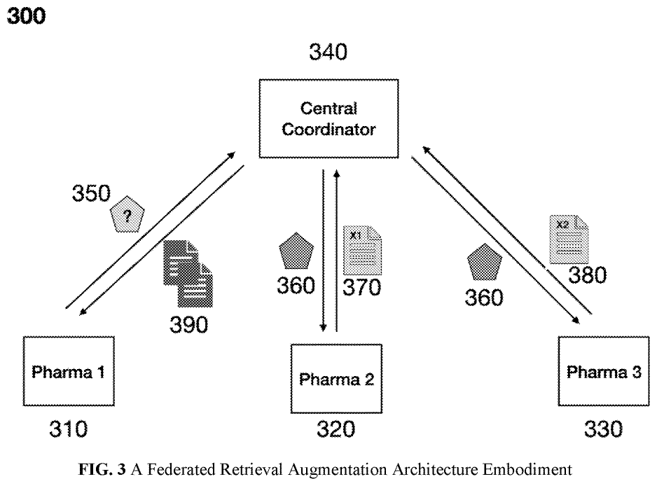

FIG. 3 is a schematic of an embodiment 300 of a federated retrieval augmentation architecture. With reference to FIG. 3, a plurality of entities 310, 320, 330 (three are shown for illustrative purposes; any number K≥2 is supported) are communicatively coupled to a central coordinator 340. The entities 310, 320, 330 may be, by way of non-limiting example, pharmaceutical companies, biotechnology companies, academic institutions, contract research organizations, or regulatory agencies; for illustrative purposes the entities are labeled Pharma 1, Pharma 2, and Pharma 3. One or more of the plurality of entities each maintains a respective local non-parametric data store comprising non-parametric data not directly shared with the other entities.

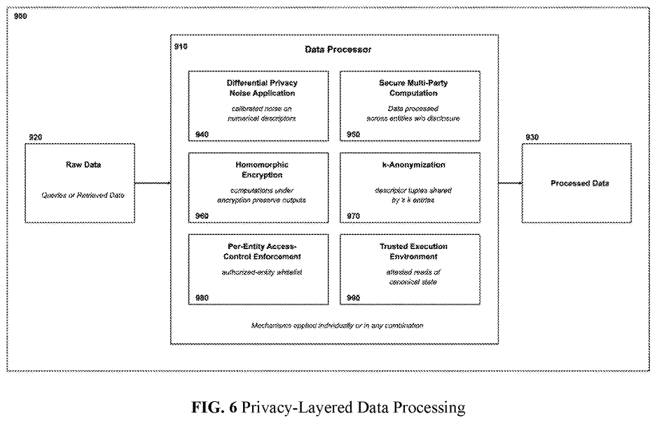

In operation, a querying entity (illustratively Pharma 1, 310) holds a query 350 for which augmentation with non-parametric data held by one or more of the other entities is sought. The query 350, or a representation thereof, is transmitted from the querying entity 310 to the central coordinator 340. The central coordinator 340 routes a representation 360 of the query 350 to one or more of the other entities (illustratively Pharma 2, 320, and Pharma 3, 330) for performance of a similarity determination between the representation of the query and respective non-parametric data held by the respective other entities. By way of non-limiting example, 360 (the representation of the query) may be a masked, encrypted, hashed, noised, or anonymized version of the query. The similarity determination is performed in a manner that neither shares the query 350 directly with any of the other entities nor directly shares the respective non-parametric data of any of the other entities with the querying entity 310, as further described in connection with FIG. 6.

In response to the representation 360 of the query, each respective other entity returns one or more similarity-matched non-parametric data objects to the central coordinator 340: For illustration purposes, a first retrieved data object 370 (illustratively designated X1) from a first other entity 320, and a second retrieved data object 380 (illustratively designated X2) from a second other entity 330. The retrieved data objects 370, 380 are aggregated by the central coordinator 340 into a retrieved data set 390 that is transmitted to the querying entity 310. The querying entity 310 then augments the query 350 with at least a portion of the retrieved data set 390 to form an augmented query, and inference is performed on a global neural network using the augmented query. The global neural network may be hosted by the central coordinator 340, by the querying entity 310, by another entity of the plurality of entities, or by a third-party computing environment.

In further embodiments, the representation 360 of the query and the retrieved data objects 370, 380 are processed via one or more privacy-enhancing mechanisms including, without limitation, differential-privacy noise application, secure multi-party computation, homomorphic encryption, k-anonymization, per-entity access-control enforcement, and a trusted execution environment with remote attestation, as further described in connection with FIG. 6. The central coordinator 340 may further be configured to authenticate the plurality of entities, to enforce access policies governing visibility of non-parametric data records, and to maintain audit logs of queries and retrievals.

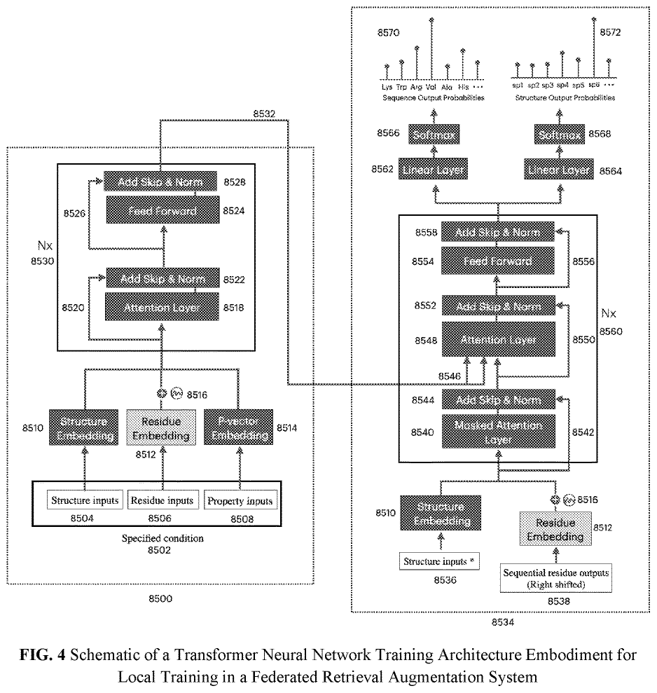

FIG. 4 is an illustrative example of a training architecture of a transformer neural network, by way of non-limiting example, with a sequence head and a structure head, accepting a specified condition comprising a structure input, a residue input, and a property input. The depicted network may be trained at a local training facility of an entity in an embodiment of the federated retrieval augmentation system disclosed herein.

With continued reference to FIG. 4, the training architecture encoder 8500 accepts a specified condition 8502 comprising a structure input 8504, a residue input 8506, and a property input 8508 into respective embedding layers: structure embedding 8510, residue embedding 8512, and a property vector (p-vector) embedding 8514. A positional encoding 8516 is added to the embedding outputs. The encoder includes an attention layer 8518 with a skip connection 8520 bypassing the attention layer, an Add Skip & Norm layer 8522, a feed forward layer 8524 with a skip connection 8526 bypassing the feed forward layer, and an Add Skip & Norm layer 8528. The encoder block module 8530 repeats N times. The encoder output passes via connection 8532 to the decoder.

The decoder 8534 receives a structure input 8536 and sequential residue outputs (right shifted) 8538. The decoder includes a masked attention layer 8540 with a skip connection 8542 bypassing the masked attention layer, an Add Skip & Norm layer 8544, a cross-attention input 8546 receiving encoder output 8532, a cross-attention layer 8548 with a skip connection 8550 bypassing the cross-attention layer, an Add Skip & Norm layer 8552, a feed forward layer 8554 with a skip connection 8556 bypassing the feed forward layer, and an Add Skip & Norm layer 8558. The decoder block module 8560 repeats N times. Above the decoder block module, a linear layer 8562 of the sequence head outputs to softmax layer 8566 to produce the sequence output probabilities 8570, and a linear layer 8564 of the structure head outputs to softmax layer 8568 to produce the structure output probabilities 8572.

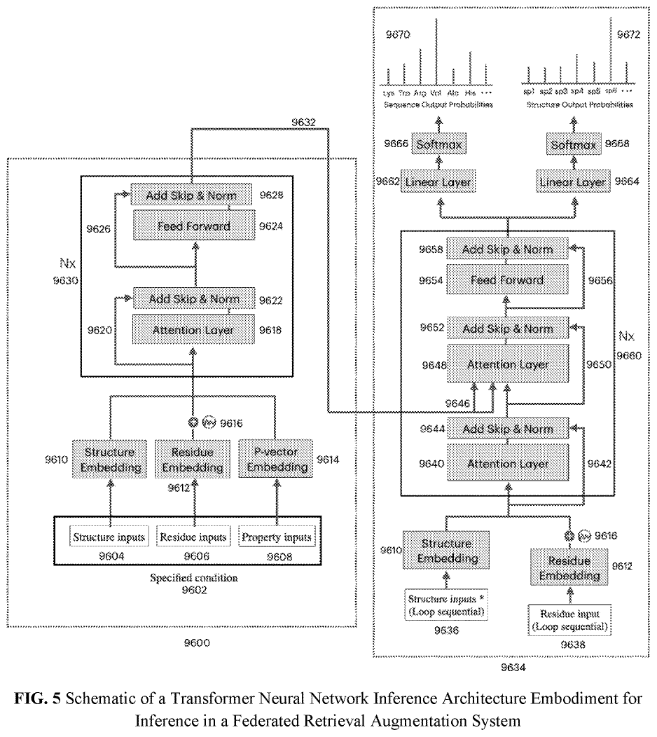

FIG. 5 is an illustrative example of an inference architecture of a transformer neural network, by way of non-limiting example, with a sequence head and a structure head, accepting a specified condition comprising a structure input, a residue input, and a property input. The depicted network embodies a trained global neural network that receives, at inference time, a query augmented in part with non-parametric data retrieved from one or more entities of the federation in accordance with the invention disclosed herein.

With continued reference to FIG. 5, the inference architecture encoder 9600 accepts a specified condition 9602 comprising a structure input 9604, a residue input 9606, and a property input 9608 into respective embedding layers: structure embedding 9610, residue embedding 9612, and p-vector embedding 9614. A positional encoding 9616 is added to the embedding outputs. The encoder includes an attention layer 9618 with a skip connection 9620 bypassing the attention layer, an Add Skip & Norm layer 9622, a feed forward layer 9624 with a skip connection 9626 bypassing the feed forward layer, and an Add Skip & Norm layer 9628. The encoder block module 9630 repeats N times. The encoder output passes via connection 9632 to the decoder.

The decoder 9634 receives a structure input (loop sequential) 9636 and a residue input (loop sequential) 9638. The decoder includes an attention layer 9640 with a skip connection 9642 bypassing the attention layer, an Add Skip & Norm layer 9644, a cross-attention input 9646 receiving encoder output 9632, a cross-attention layer 9648 with a skip connection 9650 bypassing the cross-attention layer, an Add Skip & Norm layer 9652, a feed forward layer 9654 with a skip connection 9656 bypassing the feed forward layer, and an Add Skip & Norm layer 9658. The decoder block module 9660 repeats N times. Above the decoder block module, a linear layer 9662 of the sequence head outputs to softmax layer 9666 to produce the sequence output probabilities 9670, and a linear layer 9664 of the structure head outputs to softmax layer 9668 to produce the structure output probabilities 9672.

In some embodiments of the invention disclosed herein, supplemental privacy-preserving mechanisms are employed in combination with the federated retrieval augmentation system. By way of non-limiting example, queries and/or retrieved data may be perturbed by differential-privacy noise prior to being made accessible to other entities; cross-entity flows may be protected by secure multi-party computation; inference queries and/or retrieved data may be encrypted by a homomorphic encryption scheme such that operations on the encrypted data yield the same outputs as the corresponding operations on the unencrypted data; and access-control descriptors associated with respective entries and/or entities may govern which entities of the federation are authorized to reference each non-parametric data source in response to an inference query. These supplemental mechanisms may be employed individually or in combination.

In some embodiments of the invention disclosed herein, a candidate ligand representation generated by the global neural network in response to a query is synthesized to produce a synthesized ligand. Non-limiting examples of the synthesized ligand include a small molecule, a peptide, a peptidomimetic, a cyclic peptide, an antibody, an antibody fragment, a fusion protein, an oligonucleotide, an aptamer, a proteolysis-targeting chimera (PROTAC), a molecular glue, or any other ligand modality consistent with the candidate ligand representation. The synthesized ligand is then assessed for one or more attributes selected from the group consisting of binding affinity, functional agonism, functional antagonism, biased agonism, selectivity, on-target potency, off-target activity, ADMET attributes, pharmacokinetic attributes, pharmacodynamic attributes, efficacy attributes, safety attributes, and therapeutic attributes. The assessment may be performed in vitro using one or more of binding assays, cell-based functional assays, signaling-bias assays (including without limitation assays for Gs, Gi, Gq, G12/13, beta-arrestin-1, and beta-arrestin-2 recruitment), enzymatic assays, surface plasmon resonance, isothermal titration calorimetry, hydrogen-deuterium exchange mass spectrometry, cellular thermal shift assays, and any other in vitro assays known in the art. The assessment may further or alternatively be performed in vivo in one or more animal models, including without limitation rodent models, non-human primate models, and disease-specific models.

In some embodiments of the invention disclosed herein, the synthesized ligand is administered to a human subject for therapeutic purposes, diagnostic purposes, prophylactic purposes, or research purposes. The invention disclosed herein further encompasses methods of treatment comprising administering to a subject in need thereof a therapeutically effective amount of a synthesized ligand whose candidate ligand representation was generated by a global neural network in response to a retrieval augmented query, in whole or in part, in accordance with the invention disclosed herein, the subject having a disease, disorder, or condition characterized by a target receptor conformation, a target receptor signaling profile, or a target molecular signature corresponding to the input specifications of the inference query. The invention disclosed herein further encompasses methods of diagnosis comprising assaying a sample obtained from a subject for one or more biomarkers identified by a global neural network in response to a query retrieval augmented at inference, in whole or in part, in accordance with the invention disclosed herein. The invention disclosed herein further encompasses companion-diagnostic methods comprising identifying, in response to a query retrieval augmented at inference, in whole or in part, a subject whose target conformation, signaling profile, or molecular signature corresponds to the query, and administering to the identified subject a therapeutically effective amount of a corresponding therapeutic ligand.

FIG. 6 is a schematic of an embodiment 900 of a privacy-layered data processing arrangement. With reference to FIG. 6, raw data (queries or retrieved non-parametric data) 920 is supplied as input to a data processor 910, which produces processed data 930 for distribution to subscriber entities. The data processor 910 may apply, individually or in any combination, one or more privacy-enhancing mechanisms including: differential privacy noise application 940, wherein calibrated noise is applied to one or more numerical descriptors associated with a respective data sample; secure multi-party computation 950, wherein queries or retrieved non-parametric data are processed across the plurality of entities without disclosure of underlying data; homomorphic encryption 960, wherein computations involving descriptors are performed under encryption; k-anonymization 970, wherein descriptor tuples are shared by at least k entries; per-entity access-control enforcement 980, wherein an authorized-entity whitelist governs access on a per-entry or per-entity basis; and a trusted execution environment 990 providing attested reads of canonical states. The mechanisms 940, 950, 960, 970, 980, 990 may be employed alone or in any combination thereof.

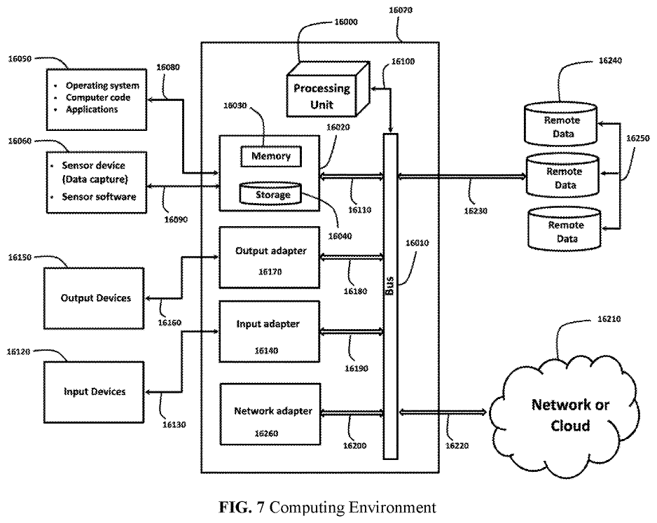

Ones with ordinary skill in the art will recognize that the invention disclosed herein can be implemented over an arbitrary range of computing configurations. We will refer to any instantiation of these computing configurations as the computing environment. An illustrative example of a computing environment is depicted in FIG. 7. Examples of computing environments include but are not limited to desktop computers, laptop computers, tablet personal computers, mainframes, mobile smart phones, smart television, programmable hand-held devices and consumer products, distributed computing infrastructures over a network, cloud computing environments, or any assembly of computing components such as memory and processing.

As illustrated in FIG. 7, the invention disclosed herein can be implemented over a system that contains a device or unit for processing the instructions of the invention. This processing unit 16000 can be a single core central processing unit (CPU), multiple core CPU, graphics processing unit (GPU), multiplexed or multiply-connected GPU system, or any other homogeneous or heterogeneous distributed network of processors.

In some embodiments of the invention disclosed herein, the computing environment can contain a memory mechanism to store computer-readable media. By way of example and not limitation, this can include removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer.

As depicted in FIG. 7, the computing environment can include a system memory 16030 which can be volatile memory such as random access memory (RAM) and may also include non-volatile memory such as read-only memory (ROM). Additionally, there typically is some mass storage device 16040 associated with the computing environment, which can take the form of hard disc drive (HDD), solid state drive, or CD, CD-ROM, blu-ray disc or other optical media storage device. In some other embodiments of the invention, the system can be connected to remote data 16240.

The computer readable content stored on the various memory devices can include an operating system, computer codes, and other applications 16050. By way of example not limitation, the operating system can be any number of proprietary software such as Microsoft Windows, Android, Macintosh operating system, iPhone operating system (iOS), or Linux commercial distributions. It can also be open source software such as Linux versions e.g. Ubuntu.

The computing environment typically includes a system bus 16010 through which the various computing components are connected and communicate with each other. The system bus 16010 can consist of a memory bus, an address bus, and a control bus. By way of example and not limitation, the processing unit 16000, storage subsystem 16020, input devices 16120, output devices 16150 can all be connected via the bus system. In the representation depicted in FIG. 7, by way of example not limitation, the processing unit 16000 can be connected to the main system bus 16010 via a bus route connection 16100; the storage subsystem 16020 can be connected via a bus route 16110; the output adapter 16170 can be connected via a bus route 16180; the input adapter 16140 can be connected via a bus route 16190; the network adapter 16260 can be connected via a bus route 16200; the remote data store 16240 can be connected via a bus route 16230; and the cloud infrastructure 16210 can be connected to the main system bus via a bus route 16220.

In some embodiments of the invention disclosed herein, the computing environment includes user input devices 16120 connected through an input adapter 16140 via an interface 16130. Examples of input devices include but are not limited to keyboards, mouse devices, stylus pens, touchscreen mechanisms, microphones, joysticks, and infrared remote control systems.

In some embodiments of the invention disclosed herein, output data, instructions, and other media can be output via any number of output devices 16150 connected through an output adapter 16170 via an interface 16160. Examples of output devices include but are not limited to computer monitors, printers, speakers, and vibration systems.

In some embodiments of the invention disclosed herein, some of the computing components can be located remotely and connected to via a wired or wireless network. A cloud 16210 and a remote data source 16240 can be connected to the main system bus 16010 via bus routes 16220 and 16230, respectively, through a network adapter 16260.

In some embodiments of the invention disclosed herein, a sensor system including a sensor device 16060 which captures and pre-processes data is attached to the system, coupled (via a connection 16090) to a storage subsystem 16020 consisting of the system memory 16030 and a mass storage 16040. The operating system, computer code, and applications 16050 are stored in the system memory 16030, accessible to the sensor device 16060 via a connection 16080. Stored in the memory mechanism—16020, 16240, or 16210—are machine learning models, algorithms, and data products developed according to the procedures set forth herein.

With continued reference to FIG. 7, the computing environment includes a computing device 16070 comprising the processing unit 16000, memory 16030, storage 16040, system bus 16010, and adapters for connecting to external entities. A storage subsystem 16020 encompasses the memory 16030 and the mass storage device 16040. Additionally, the remote data storage infrastructure 16240 may include a plurality of remote data stores connected via connector 16250.

The objects set forth in the preceding are presented in an illustrative manner for reason of efficiency. It is hereby noted that the above-disclosed methods and systems can be implemented in manners such that modifications are made to the particular illustration presented above, while yet the spirit and scope of the invention is retained. The interpretation of the above disclosure is to contain such modifications, and is not to be limited to the particular illustrative examples and associated drawings set-forth herein.

Furthermore, by intention, the following claims encompass all of the general and specific attributes of the invention described herein; and encompass all possible expressions of the scope of the invention, which can be interpreted—as pertaining to language—as falling between the aforementioned general and specific ends.

1. A system for performing an AI-based drug discovery and development task, the system comprising:
a) a plurality of entities, one or more of the plurality of entities each maintaining a respective local non-parametric data store comprising respective non-parametric data;
b) a coordinator communicatively coupled to the plurality of entities, the coordinator configured to:
i) receive, from a respective one of the plurality of entities, a query;
ii) cause a similarity determination to be performed between the query and the respective non-parametric data of each of the one or more other entities of the plurality of entities; and
iii) transmit, to the respective one of the plurality of entities, at least one retrieved non-parametric data object selected from the respective non-parametric data based on the respective similarity determinations; and
c) a global neural network configured to receive an augmented query comprising the query augmented with at least a portion of the at least one retrieved non-parametric data object, and to produce an inference output supporting the AI-based drug discovery and development task:
i) wherein the query is not directly transferred from the respective one of the plurality of entities submitting the query to any of the one or more other entities of the plurality of entities,
ii) and wherein the respective non-parametric data of any of the one or more other entities of the plurality of entities is not directly transferred to the respective one of the plurality of entities submitting the query.

2. The system of claim 1, wherein each respective one of the plurality of entities is selected from the group consisting of: a pharmaceutical company, a biotechnology company, an academic institution, a contract research organization, a contract development and manufacturing organization, and a regulatory agency.

3. The system of claim 1, wherein the global neural network was trained on training data that excludes the respective non-parametric data of the plurality of entities, and wherein augmentation of the query with the at least one retrieved non-parametric data object enables the global neural network to produce the inference output in dependence on the respective non-parametric data without the respective non-parametric data being included in training data of the global neural network.

4. The system of claim 1, further comprising one or more privacy-enhancing mechanisms applied to one or more of the query, the respective non-parametric data, and the at least one retrieved non-parametric data object, the one or more privacy-enhancing mechanisms selected from the group consisting of: differential privacy, secure multi-party computation, homomorphic encryption, k-anonymization, per-entity access control, and a trusted execution environment with remote attestation.

5. The system of claim 1, wherein the coordinator comprises one or more of: a central server, a distributed set of servers, a peer-to-peer protocol executed by the plurality of entities, a trusted execution environment hosted by one of the plurality of entities or by a third party, or a smart contract executed on a blockchain or other distributed ledger.

6. The system of claim 1, wherein the AI-based drug discovery and development task comprises generation of a candidate ligand representation for a target protein.

7. The system of claim 6, wherein the target protein comprises one or more of: a G-Protein Coupled Receptor (GPCR), an ion channel, an enzyme, a nuclear receptor, a receptor tyrosine kinase (RTK), a transporter, a cytokine receptor, or a Toll-like receptor.

8. A method for performing an AI-based drug discovery and development task, the method comprising:
a) receiving, by a coordinator communicatively coupled to a plurality of entities, from a respective one of the plurality of entities, a query, one or more of the plurality of entities each maintaining a respective local non-parametric data store comprising respective non-parametric data;
b) causing, by the coordinator, a similarity determination to be performed between the query and the respective non-parametric data of each of the one or more other entities of the plurality of entities;
c) transmitting, by the coordinator to the respective one of the plurality of entities, at least one retrieved non-parametric data object selected from the respective non-parametric data based on the respective similarity determinations;
d) augmenting the query with at least a portion of the at least one retrieved non-parametric data object to form an augmented query;
e) performing inference on a global neural network using the augmented query to produce an inference output; and
f) using the inference output to support the AI-based drug discovery and development task:
i) wherein the query is not directly transferred from the respective one of the plurality of entities submitting the query to any of the one or more other entities of the plurality of entities,
ii) and wherein the respective non-parametric data of any of the one or more other entities of the plurality of entities is not directly transferred to the respective one of the plurality of entities submitting the query.

9. The method of claim 8, wherein each respective one of the plurality of entities is selected from the group consisting of: a pharmaceutical company, a biotechnology company, an academic institution, a contract research organization, a contract development and manufacturing organization, and a regulatory agency.

10. The method of claim 8, wherein the global neural network was trained on training data that excludes the respective non-parametric data of the plurality of entities, and wherein augmentation of the query with the at least one retrieved non-parametric data object enables the global neural network to produce the inference output in dependence on the respective non-parametric data without the respective non-parametric data being included in training data of the global neural network.

11. The method of claim 8, further comprising applying one or more privacy-enhancing mechanisms to one or more of the query, the respective non-parametric data, and the at least one retrieved non-parametric data object, the one or more privacy-enhancing mechanisms selected from the group consisting of: differential privacy, secure multi-party computation, homomorphic encryption, k-anonymization, per-entity access control, and a trusted execution environment with remote attestation.

12. The method of claim 8, wherein the AI-based drug discovery and development task comprises generation of a candidate ligand representation for a target protein.

13. The method of claim 12, wherein the target protein comprises one or more of: a GPCR, an ion channel, an enzyme, a nuclear receptor, a receptor tyrosine kinase (RTK), a transporter, a cytokine receptor, or a Toll-like receptor.

14. The method of claim 12, further comprising synthesizing a ligand from the candidate ligand representation, the ligand selected from the group consisting of: a small molecule, a peptide, a peptidomimetic, a cyclic peptide, a stapled peptide, an antibody, an antibody fragment, a fusion protein, a nucleic-acid-based ligand, an oligonucleotide, an aptamer, a proteolysis-targeting chimera (PROTAC), and a molecular glue.

15. A method of manufacturing a ligand, the method comprising:
a) receiving, by a coordinator communicatively coupled to a plurality of entities, from a respective one of the plurality of entities, a query;
b) causing, by the coordinator, a similarity determination to be performed between the query and respective non-parametric data respectively held by each of one or more other entities of the plurality of entities;
c) transmitting, by the coordinator to the respective one of the plurality of entities, at least one retrieved non-parametric data object selected from the respective non-parametric data based on the respective similarity determinations;
d) augmenting the query with at least a portion of the at least one retrieved non-parametric data object to form an augmented query;
e) generating, by a global neural network, a candidate ligand representation in response to the augmented query; and
f) synthesizing a ligand corresponding to the candidate ligand representation, thereby producing a synthesized ligand:
i) wherein the query is not directly transferred from the respective one of the plurality of entities submitting the query to any of the one or more other entities of the plurality of entities,
ii) and wherein the respective non-parametric data of any of the one or more other entities of the plurality of entities is not directly transferred to the respective one of the plurality of entities submitting the query.

16. The method of claim 15, further comprising assessing a biological activity of the synthesized ligand in vitro or in vivo.

17. The method of claim 15, further comprising administering the synthesized ligand to a subject for one or more of: therapeutic, prophylactic, diagnostic, or research purposes.

18. The method of claim 16, further comprising in vitro signaling-bias assessment of the synthesized ligand at one or more GPCR signaling pathways, the signaling pathways comprising one or more of: Gs, Gi, Gq, G12/13, beta-arrestin-1 recruitment, or beta-arrestin-2 recruitment.

19. The method of claim 15, wherein the synthesized ligand is selected from the group consisting of: a small molecule, a peptide, a peptidomimetic, a cyclic peptide, a stapled peptide, an antibody, an antibody fragment, a fusion protein, a nucleic-acid-based ligand, an oligonucleotide, an aptamer, a proteolysis-targeting chimera (PROTAC), and a molecular glue.

20. The method of claim 15, wherein the synthesized ligand is a CXCR4 ligand.
